(19) United States
(12) Patent Application Publication
(10) Pub. No.: US 2023/0414732 A1
(43) Pub. Date: Dec. 28, 2023

(54) COMPOSITIONS OF EMBEDDED EPITOPE RANDOM PEPTIDES (EERP) FOR TREATMENT OF IMMUNE-MEDIATED CONDITIONS, AND METHODS OF USE

(71) Applicant: PALENA THERAPEUTICS, INC., Worcester, MA (US)

(72) Inventor: Masha FRIDKIS-HARELI, Sudbury, MA (US)

(73) Assignee: PALENA THERAPEUTICS, INC., Worcester, MA (US)

(21) Appl. No.: 18/035,837

(22) PCT Filed: Nov. 8, 2021
(86) PCT No.: PCT/US2021/058379

§ 371 Date: May 8, 2023

Related U.S. Application Data
(60) Provisional application No. 63/111,746, filed on Nov. 10, 2020.

(51) Int. Cl. A61K 39/00; C07K 14/00; A61P 37/06

(52) U.S. Cl. CPC A61K 39/0008; C07K 14/001; A61P 37/06; A61K 2039/577

(57) ABSTRACT

Embedded Epitope Random Peptides (EERP) for the treatment of autoimmune diseases are provided. Each EERP is a polypeptide consisting of a random sequence of three or more amino acids in a specific molar ratio, within which is embedded an epitope composed of a specific amino acid sequence. Also provided is a method of treating an autoimmune disease or condition using the EERP.

### RELATED APPLICATIONS

The present application claims priority from U.S. Provisional Patent Application Ser. No. 63/111746, filed on Nov. 10, 2020, the disclosure of which incorporated herein by reference in its entirety.

### TECHNICAL FIELD

The invention relates to polypeptides having an epitope of known amino acid sequence embedded into a random amino acid sequence. The amino acids constituting the random amino acid sequence are present in specific molar ratios. The polypeptides are capable of modifying an immune response, such as suppressing symptoms and/or frequency of episodes of various immune-mediated conditions.

### BACKGROUND ART

The immune system, under normal circumstances, is able to respond to a vast array of foreign materials, but not to self-antigens. This leads to tolerance of the self. Although recognition of self plays an important role in generating both the T cell and B cell repertoires, and plays an essential role in the recognition of antigens by T cells, the development of potentially harmful response to self-antigens is, in general, precluded. Autoimmunity results from the breakdown of one or more of the basic mechanisms regulating tolerance to self-antigens. An essential feature of an autoimmune disease is injury caused by immunologic reaction by the organism against its own tissue.

Autoimmune diseases display a spectrum of clinical manifestations ranging from effects on a single organ to effects on multiple organs (i.e., a systemic disorder). Examples of the former include ankylosing spondylitis and related spondyloarthropathies, autoimmune polyglandular syndrome, autoimmune Addison's disease, autoimmune alopecia, autoimmune hemolytic anemia, autoimmune thrombocytopenic purpura, acute rheumatic fever, age-related macular degeneration, celiac sprue, Crohn's disease, inflammatory bowel disease, dense deposit disease, dermatitis herpetiformis, Grave's disease, Goodpasture's syndrome, Guillain-Barre syndrome, Hashimoto's thyroiditis, immune-mediated infertility, insulin-dependent diabetes mellitus, insulin-resistant diabetes mellitus, multiple sclerosis, myasthenia gravis, neuromyelitis optica, pemphigus foliaceus, pemphigus vulgaris, rosacea, pernicious anemia, stiff-man syndrome, sympathetic ophthalmia, and vitiligo. Examples of systemic diseases include antiphospholipid syndrome, obstetrical antiphospholipid syndrome, rheumatoid arthritis, Sjogren's syndrome, systemic lupus erythematosus, systemic necrotizing vasculitis, autoimmune vasculitis, Wegener's granulomatosis (Braunwald E. et al., Principles of Internal Medicine, 15th edition, McGraw-Hill eds. (2001)). In addition, there are immune-mediated disorders of infectious origin (e.g., sepsis and septic shock, necrotizing enterocolitis, hemorrhagic fevers (e.g., Ebola, dengue and others)); nephrological conditions, e.g., CFHR5 nephropathy, delayed graft-function, renal ischemia-reperfusion injury, IgA nephropathy (Berger's disease), antibody-mediated rejection, E-coli shiga toxin mediated disease (mostly kidney damage by toxin), and atypical HUS; hematological disorders (e.g., disseminated intravascular coagulation, deep vein thrombosis, warm autoimmune hemolytic anemia, Glanzmann's thrombasthenia, and sickle cell anemia); preeclampsia; and asthma.

Cell surface proteins known as major histocompatibility complex (MHC) proteins in mammals (human leukocyte antigens, HLA in humans) play a central role in the regulation of immune response through their ability to bind and present processed peptides to T cells. (Rothbard, J. B., et al., 1991, Annu. Rev. Immunol. 9:527). These genes are located on chromosome 6p21, which is a principal susceptibility locus for many human autoimmune diseases. MHC is a multi-gene family of antigen receptors, which play a key role in the selection and establishment of the antigen-specific T cell repertoire and the subsequent activation of the T cells during the initiation of an immune response. The MHC region encodes genes for class I, II, and III proteins which are expressed differentially in various cells of the body. Class I gene region is subdivided into A, B, and C subregions. Class II gene region consists of DR, DQ, and DP subregions, each expressed on the surface of APC such as macrophages, B cells, dendritic cells of lymphoid tissue, and epidermal cells. Class III gene products are expressed in various components of the complement system, as well as in some non-immune related cells. Each of the MHC genes is found in a large number of alternative or allelic forms within a mammalian population. The genomes of subjects affected with certain autoimmune diseases are more likely to carry one or more characteristic MHC alleles, to which that disease is linked.

Structural characteristics of MHC molecules, including those of the disease-linked alleles, dictate the specificity of their interactions with T cells, and thereby instruct and guide antigen-specific immune events. The primary role of the MHC molecule, including during the onset of an immune response, is to bind and present a peptide to T cells. Peptides that bind to MHC molecules are capable of stimulating T cell responses and, hence, are antigenic; peptides that do not bind are not presented to T cells and are not antigenic. Polymorphic sites present within the MHC peptide-binding groove influence binding to peptides and as such, the MHC complex can be viewed as encoding genetic determinants of precise immunological activation events (Rothbard J. B. et al., Annu. Rev. Immunol. 9:527 (1991); Gebe J. A. et al., Tissue Antigens 59:78 (2002)).

In autoimmune diseases, it is likely that tissue that are targets of pathogenic T cells express MHC molecules encoded by specific susceptibility alleles. In autoimmune diseases with the infectious etiology, it is likely that immune responses to peptides derived from the initiating pathogen are bound and presented by particular MHC molecules to activate T lymphocytes that play a triggering or contributory role in disease pathogenesis. The concept that early events in disease initiation are triggered by specific MHC-peptide complexes offers some prospects for therapeutic intervention, since it may be possible to design compounds that interfere with the formation or function of specific MHC-peptide—TCR interactions. Inhibition of antigen presentation by disease-associated MHC molecules could interfere with the autoimmune process. This inhibition can be based on binding to an MHC molecule of peptides that have certain specific side chains at anchor positions, but that allow for a large variety of side chains at non-anchor positions, and the presentation of these peptides to T cells.

It should be noted that several strategies for inducing immunological tolerance have been reported. These include blocking antigen presentation, supplying altered peptide ligands, developing tolerance by intravenous and/or oral administration, and blocking costimulatory molecules (Sakai K. et al., 1989. Proc. Natl. Acad. Sci. U.S.A. 86:9470; Hurtenbach U. et al., 1993. J. Exp. Med. 177:1499; Fairchild P. J. et al., 1994. Immunology 81:487; Brocke S. et al., 1996. Nature 379:343).

A number of therapeutic agents have been developed to treat autoimmune diseases, including general anti-inflammatory drugs such as “super aspirins”, for example, agents that can prevent formation of low molecular weight inflammatory compounds by inhibiting a cyclooxygenase; various types of interferons; and inhibitors of prostaglandin synthesis. However, these agents can be toxic when used for more than short periods of time or exhibit undesirable side effects. Other therapeutic agents function by inhibiting a protein mediator of inflammation, for example, by sequestering the inflammatory protein tumor necrosis factor (TNF) with an anti-TNF specific monoclonal antibody or antibody fragment, or with a soluble form of the TNF receptor; agents that target a protein on the surface of a T cell and generally prevent interaction with an antigen presenting cell (APC) by inhibiting the CD4 receptor or the cell adhesion receptor ICAM-1. However, compositions having proteins—which must have proper folding—as therapeutic agents can lead to problems in production, formulation, storage, and delivery. Several of these problems necessitate delivery of the therapeutic agent to the patient in a hospital setting.

Improved treatments for autoimmune disease can potentially come from the identification of agents that bind selectively to a purified MHC class I or class II protein molecules in vitro. Such MHC protein is the product of the MHC allele associated with the autoimmune disease for which a treatment is being sought. The agent should be capable also of binding to the protein as it occurs on the surfaces of APC in vivo, and thus block, anergize, inhibit, inactivate or reprogram T cells that are responsible for mediating the autoimmune disease.

An agent that interacts and binds relatively nonspecifically to several MHC class II molecules is Copolymer 1 (Cop 1), a synthetic amino acid copolymer that was shown to be capable of suppressing experimental allergic encephalomyelitis (EAE; Sela, M., R. Amon, et al., 1990. Bull. Inst. Pasteur (Paris) 88:303), which can be induced in a mouse model for multiple sclerosis (MS). Cop 1 (Copaxone®) which is poly (Y, E, A, K), indicated herein “YEAK” using the one letter amino acid code (see infra; Y represents tyrosine, E glutamic acid, A alanine, and K lysine) has been used to treat relapsing forms of MS but does not suppress the disease entirely, resulting in about 30% reduction in relapses (Bornstein, M. B., et al., 1987, N. Engl. J. Med. 317:408; Johnson, K. P., et al., 1995, Neurology 45:1268). More recently, substitutions of amino acids in the composition of Copaxone® resulted in random copolymers that suppressed EAE more efficiently (Fridkis-Hareli M, et al., 2002, .J Clin Invest. 109:1635) and inhibited collagen type II-reactive T cells in an ex-vivo model for RA (Fridkis-Hareli M, et al., 1998, Proc Natl Acad Sci U S A 95:12528). In addition, peptides designed based on the binding motifs of Copaxone® to class II MHC molecules associated with MS and RA (Fridkis-Hareli M, et al., 1999, Immunol. 162:4697) were shown to inhibit binding of MBP85-99 (Fridkis-Hareli et al., 2001, Hum Immunol. 62:753), of collagen type II 261-273 autoantigenic peptides (Fridkis-Hareli M, et al., 2000, Hum Immunol. 61:640) and of their respective autoreactive T cells.

There is a need for improved treatments for autoimmune diseases that are more efficacious than Copaxone and that at the same time have minimal side effects as compared to current disease modifying immunotherapies. Longer relapse-free intervals will delay the disease progression and thus ultimately improve the quality of life in these patients.

### SUMMARY

The present invention provides Embedded Epitope Random Peptides (EERP) for the treatment of autoimmune diseases. An EERP is a polypeptide consisting of two polypeptides, one consisting of a random sequence of three or more amino acids in a specific molar ratio, and a second polypeptide that defines an epitope composed of a specific amino acid sequence embedded within the polypeptide of random amino acid sequence. An EERP has a fixed length and is capable of suppressing symptoms and/or frequency of recurrent episodes of an autoimmune or immune-mediated disease when bound to an MHC class I or class II molecule associated with a disease. The EERP is explained in greater detail below.

The epitope is peptide sequence, assembled based on occupancy of certain anchor positions of an MHC class I or class II molecule by amino acids of a peptide bound to the MHC molecule in the context of an autoimmune disease or condition.

Referring to the structure of EERP that can bind to MHC class II molecules, the structure is such that in addition to binding to the MHC molecule, there is interaction between the amino acid residues of the EERP and the T cell receptor that forms a complex with the EERP bound to the MHC molecule. Proper positioning of the epitope within the random linear polypeptide leads to better fit and more specific binding of the EERP in the binding groove of the MHC molecule which leads to inhibition of interaction of disease-associated MHC gene products with self-tissue antigens (autoantigens). As a result of this binding, further exposure of autoantigens to autoreactive and cross-reactive T cells—which is harmful to the individuals affected with the autoimmune condition through damage to self-organs—is prevented or severely reduced. Further, EERP modulate T cell reactivity by interacting with the T cell receptors (TCR), leading to irresponsiveness of pathogenic T cells. Additionally, EERP have the potential of reprogramming T cell responses from pro-inflammatory to anti-inflammatory at the APC level (monocyte/macrophage shift from M1 to M2 type, as determined by the cytokine/chemokine profile and immunophenotyping of surface receptors, or effects on autoreactive B cells). Thus, EERP interfere with the complex mechanisms involved in pathogenic processes and represent potential therapies for a wide range of immune-mediated diseases. Due to their novel composition having an epitope of specific amino acids that can fit into the binding site of many MHC molecules, EERP are beneficial to any individual affected with specific immune-mediated disease, or multiple diseases. From a therapeutic standpoint, even though EERP can bind to many MHC molecules they selectively down regulate the immune response to specific antigen targets unlike broad spectrum therapeutics that can down regulate the entire immune system which is problematic in fighting off common infections.

The EERP may be composed of naturally occurring amino acids such as Ala, Gly, Ile, Leu, Met, Phe, Pro, Trp, Tyr, and Val. The EERPs may also incorporate alternative amino acids, such as D-amino acids as well as modified amino acids, e.g., amino acids modified by amidation (to increase half-life or prevent protease degradation) (Erak M. et al., 2018, Bioorg. Med. Chem. 26:2759). The amino acids may be polymerized by a solid phase reaction; alternatively, they may be polymerized by solution chemistry.

Accordingly, in one aspect, provided herein is an amino acid copolymer (EERP) having the sequence

(X)₀₋₁₉₅(Z)₅₋₂₀(X)₀₋₁₉₅

wherein X is one of tyrosine(Y), phenylalanine(F), alanine(A), and lysine(K); and Z consists of a peptide epitope that interacts with a major histocompatibility complex (MHC) class II protein and a T cell receptor.

In some embodiments, the peptide epitope has the amino acid sequence AYKAAA. In the EERP, Y, F, A, and K may be present in a molar ratio of about 0.01-3:0.01-5:2-30:1-10, respectively.

In a preferred embodiment, the EERP is between 30 and 200 amino acids in length with the epitope positioned between 5 and 20 amino acids. The preferred start position of the epitope is within the random polypeptide is at residue 7 from the N-terminus, however, it may start at any residue from the N-terminus. For example, from residue 1 through residue X as long as the length of the epitope fits within the total length of the polypeptide.

In one aspect, provided herein is a method for identifying a therapeutic EERP capable of reducing severity and frequency of episodes of an autoimmune disease. The method comprises: (a) providing a plurality of types of amino acids to serve as component amino acid for polymerization, the amino acids having properties such as size, charge and hydrophobicity of side chains such that the peptide produced can bind to (i) binding groove of the protein of an MHC class II allele associated with the autoimmune disease, and (ii) a T cell receptor; (b) polymerizing the amino acids in a plurality of different molar ratios by solid phase chemistry or in solution, thereby obtaining a set of resulting polymers, each member of the set of resulting polymers having different molar ratios of component amino acids, and being of a predetermined number of amino acid residues; and (c) testing the resulting polypeptides in vitro with purified molecules of the class II MHC allele protein, autoantigenic peptide and T cells, or in cell-based assays by incubating the EERP with the primary immune cells or with cell lines and measuring cellular function, and optionally, in vivo in a subject susceptible to the autoimmune disease, to identify types of amino acids, the molar ratios, and the length of EERP having efficacy in reducing severity and frequency of an episode of the disease, compared to an untreated subject having the autoimmune disease.

In one aspect, provided herein is an assay for testing sets of EERP in vitro. The method comprises measuring binding of autoantigenic peptide and EERP to an MHC class II protein competitively, competitive binding to living antigen-presenting cells expressing class II MHC protein, inhibition of proliferation of T cells following presentation of the EERP by an antigen presenting cell (APC) and/or incubating EERP with the primary cells or cell lines and accessing their functionality by measuring cytokine and surface receptor profiling. Further, testing the EERP in vivo is accomplished by administering each of the EERP to a non-human subject susceptible to an experimentally induced immune-mediated disease. The non-human subject, for example, is a mouse susceptible to induction of an experimental immune-mediated disease. Testing the EERP is assessing their effect on development of symptoms associated with experimental disease.

In another embodiment, the invention provides a method of treating a subject having an immune-mediated disease, comprising: a) selecting a therapeutic EERP comprising: at least three different amino acids, the amino acids being polymerized in a linear configuration, and a pharmaceutically acceptable carrier; and b) administering the therapeutic EERP and carrier to the subject having the immune-mediated disease.

In another related embodiment, step (a) comprises selecting the EERP that inhibits binding of a high affinity peptide to an MHC class II protein. In another related embodiment, step (a) further comprises selecting the EERP that inhibits a class II-specific T cell response to an MHC class II protein-peptide complex. The autoantigenic peptide is associated with an autoimmune disease. In another related embodiment, the class II MHC protein is associated with the autoimmune disease.

A feature of the invention is a method of designing and manufacturing of a composition comprising EERP for use in treating a subject having an immune-mediated disease. The epitope within the EERP is composed of naturally occurring amino acids. In general, EERP has a length of at least about amino acids to 200 amino acids. Further, in such a use, the composition comprises a pharmaceutically acceptable carrier.

Further, the use can involve administering the composition in an effective dose. An “effective dose” is an amount of the composition that remediates either or both of clinical symptoms and frequency of recurrence of an immune-mediated disease. Prior to administering, the EERP is selected for inhibiting binding of a high affinity binding peptide to an MHC class II protein associated with the immune-mediated disease. Further, the EERP that inhibits a class II-specific T cell response to an MHC class II protein-peptide complex is selected.

In one aspect, one or more amino acids of the EERP is modified. For example, the N-terminal amino acid or the C-terminal amino acid of the EERP or both the N-terminal and the C-terminal of the EERP may be modified. The N-terminal amino acid of the EERP may modified, for example, with an acetyl group and the C-terminal amino acid may be modified, for example, with an amide group. In addition, other amino may also be modified to improve the properties of the EERP such as to inhibit proteolytic degradation of the copolymer in a subject compared to a copolymer which is otherwise identical but lacking the amino acid modification.

In one aspect, the EERP is provided in combination with at least one additional therapeutic agent. The additional therapeutic agent may be an antibody, an enzyme inhibitor, an antibacterial, an antiviral, a steroid, a nonsteroidal anti-inflammatory, an antimetabolite, a cytokine, a cytokine blocking agent, an adhesion molecule blocking agent, or a soluble cytokine receptor. The cytokine may be β-interferon, interleukin-4, or interleukin-10.

In one aspect, the EERP is provided in a composition a pharmaceutically acceptable carrier.

In one aspect, provided herein is a method of treating an immune-mediated disorder or an autoimmune disease in a subject in need thereof. The method comprises administering to the subject a therapeutically effective amount of the above composition. The immune-mediated disorder or an autoimmune disease may be one of the following: ankylosing spondylitis and related spondyloarthropathies, autoimmune polyglandular syndrome, autoimmune Addison's disease, autoimmune alopecia, autoimmune hemolytic anemia, autoimmune thrombocytopenic purpura, acute rheumatic fever, age-related macular degeneration, celiac sprue, Crohn's disease, inflammatory bowel disease, dense deposit disease, dermatitis herpetiformis, Grave's disease, Goodpasture's syndrome, Guillain-Barre syndrome, Hashimoto's thyroiditis, immune-mediated infertility, insulin-dependent diabetes mellitus, insulin-resistant diabetes mellitus, multiple sclerosis, myasthenia gravis, neuromyelitis optica, pemphigus foliaceus, pemphigus vulgaris, rosacea, pernicious anemia, stiff-man syndrome, sympathetic ophthalmia, vitiligo, antiphospholipid syndrome, obstetrical antiphospholipid syndrome, rheumatoid arthritis, Sjogren's syndrome, systemic lupus erythematosus, systemic necrotizing vasculitis, autoimmune vasculitis, Wegener's granulomatosis, sepsis and septic shock, necrotizing enterocolitis, hemorrhagic fevers (e.g., Ebola, dengue and others), CFHR5 nephropathy, delayed graft-function, renal ischemia-reperfusion injury, IgA nephropathy (Berger's disease), antibody-mediated rejection, E-coli shiga toxin mediated disease (mostly kidney damage by toxin), atypical hemolytic uremic syndrome (aHUS, disseminated intravascular coagulation, deep vein thrombosis, warm autoimmune hemolytic anemia, Glanzmann's thrombasthenia, sickle cell anemia, preeclampsia, and asthma.

A “therapeutically effective amount” is an amount of the composition that remediates either or both of clinical symptoms and frequency of recurrence of an immune-mediated disease. Prior to administering, the EERP is selected for inhibiting the binding of an antigen associated with an autoimmune disease to the MHC class II protein. Further, the EERP that inhibits a class II-specific T cell response to an MHC class II protein-peptide complex is selected.

In one aspect, provided herein is a kit comprising at least one unit dosage of the above-described EERP.

The invention is further summarized below by the following list of embodiments.

In a 1^{st} embodiment, provided herein is an amino acid copolymer having the sequence

(X)₀₋₁₉₅(Z)₅₋₂₀(X)₀₋₁₉₅

wherein X is one of tyrosine(Y), phenylalanine(F), alanine(A), and lysine(K); and Z consists of a peptide epitope that interacts with a major histocompatibility complex (MHC) class II protein and a T cell receptor.

In a 2^{nd} embodiment, provided herein is the copolymer of the 1st embodiment, wherein the peptide epitope has the amino acid sequence AYKAA.

In a 3^{rd} embodiment, provided herein is the copolymer of the 1^{st} and 2^{nd} embodiments, wherein Y, F, A, and K are present in a molar ratio of about 0.01-3:0.01-5:2-30:1-10, respectively.

In a 4^{th} embodiment, provided herein is the copolymer of the 1^{st} and 2^{nd} embodiments, wherein Y, F, A, and K are present in a molar ratio of about 0.1-2.5:0.1-5.0:2-25:2-8, respectively.

In a 5^{th} embodiment, provided herein is the copolymer of the 1^{st} and 2^{nd} embodiments, wherein Y, F, A, and K are present in a molar ratio of about 0.8-2.0:0.1-4.0:2-20:2-6, respectively.

In a 6^{th} embodiment, provided herein is the copolymer of the 1^{st} and 2^{nd} embodiments, wherein Y, F, A, and K are present in a molar ratio of about 0.5-1.5:0.8-3.0:2-15:2-5, respectively.

In a 7^{th} embodiment, provided herein is the copolymer of the 1^{st} and 2^{nd} embodiments, wherein, wherein Y, F, A, and K are present in a molar ratio of about 0.2-1.0:0.8-2.0:2-10:2-4, respectively.

In an 8^{th} embodiment, provided herein is the copolymer of the 1^{st} and 2^{nd} embodiments The copolymer of claim 1 or 2, wherein Y, F, A, and K are present in a molar ratio of about 0.2-0.5:0.6-1.0:5-10:3-4, respectively.

In a 9^{th} embodiment, provided herein is the copolymer of the 1^{st} and 2^{nd} embodiments, wherein Y, F A, and K are present in a molar ratio of about 0.2:0.5:5:3, respectively.

In a 10^{th} embodiment, provided herein is the copolymer of any of the preceding embodiments, wherein the copolymer comprises at least about 30 amino acid residues.

In an 11^{th} embodiment, provided herein is the copolymer of any of the preceding embodiments, wherein the copolymer comprises between about 30 and 200 and amino acid residues.

In a 12^{th} embodiment, provided herein is the copolymer of any of the preceding embodiments, wherein the amino acids are polymerized using a solid phase reaction.

In a 13^{th} embodiment, provided herein is the copolymer of any of the preceding embodiments, wherein the N-terminal amino acid is modified with an acetyl group.

In a 14^{th} embodiment, provided herein is the copolymer of any of the preceding embodiments, wherein the C-terminal amino acid is modified with an amide group.

In a 15^{th} embodiment, provided herein is the copolymer of any of the preceding embodiments, wherein one or more amino acids are modified to inhibit proteolytic degradation of the copolymer in a subject compared to a copolymer which is otherwise identical but lacking the amino acid modification.

In a 16^{th} embodiment, provided herein is the copolymer of any of the preceding embodiments, combined with at least one additional therapeutic agent.

In a 17^{th} embodiment, provided herein is the copolymer of the 16^{th} embodiment, wherein the additional therapeutic agent is selected from the group consisting of an antibody, an enzyme inhibitor, an antibacterial, an antiviral, a steroid, a nonsteroidal anti-inflammatory, an antimetabolite, a cytokine, a cytokine blocking agent, an adhesion molecule blocking agent, and a soluble cytokine receptor.

In an 18^{th} embodiment, provided herein is the copolymer of the 17^{th} embodiment, wherein the cytokine is selected from the group consisting of β-interferon, interleukin-4, and interleukin-10.

In an 19^{th} embodiment, provided herein is a composition comprising the copolymer of any of the preceding embodiments and a pharmaceutically acceptable carrier.

In an 20^{th} embodiment, provided herein is a method of treating an immune-mediated disorder or an autoimmune disease in a subject in need thereof, the method comprising administering to the subject a therapeutically effective amount of the composition of the 19^{th} embodiment.

In a 21^{th} embodiment, provided herein is a method of the 20^{th} embodiment, wherein the immune-mediated disorder or an autoimmune disease is selected from the group consisting of ankylosing spondylitis and related spondyloarthropathies, autoimmune polyglandular syndrome, autoimmune Addison's disease, autoimmune alopecia, autoimmune hemolytic anemia, autoimmune thrombocytopenic purpura, acute rheumatic fever, age-related macular degeneration, celiac sprue, Crohn's disease, inflammatory bowel disease, dense deposit disease, dermatitis herpetiformis, Grave's disease, Goodpasture's syndrome, Guillain-Barre syndrome, Hashimoto's thyroiditis, immune-mediated infertility, insulin-dependent diabetes mellitus, insulin-resistant diabetes mellitus, multiple sclerosis, myasthenia gravis, neuromyelitis optica, pemphigus foliaceus, pemphigus vulgaris, rosacea, pernicious anemia, stiff-man syndrome, sympathetic ophthalmia, vitiligo, antiphospholipid syndrome, obstetrical antiphospholipid syndrome, rheumatoid arthritis, Sjogren's syndrome, systemic lupus erythematosus, systemic necrotizing vasculitis, autoimmune vasculitis, Wegener's granulomatosis, sepsis and septic shock, necrotizing enterocolitis, hemorrhagic fevers (e.g., Ebola, dengue and others), CFHR5 nephropathy, delayed graft-function, renal ischemia-reperfusion injury, IgA nephropathy (Berger's disease), antibody-mediated rejection, E-coli shiga toxin mediated disease (mostly kidney damage by toxin), atypical hemolytic uremic syndrome (aHUS, disseminated intravascular coagulation, deep vein thrombosis, warm autoimmune hemolytic anemia, Glanzmann's thrombasthenia, sickle cell anemia, preeclampsia, and asthma.

In a 22^{st} embodiment, provided herein is a kit comprising at least one unit dosage of the copolymer of any of claims 1-18.

### BRIEF DESCRIPTION OF DRAWINGS

FIG. 1 depicts the design of a specific Embedded Epitope Random Peptide (EERP) according to the present invention. The EERP is a polypeptide consisting of two polypeptides: a first polypeptide consisting of a random sequence of three or more amino acids in a specific molar ratio, each amino acid occurring multiple times; and a second polypeptide defining an epitope of a specific amino acid sequence embedded within the first polypeptide. In the embodiment shown in this FIG. 1, X stands for a mixture of Y, F, A, and K at the molar ratio of 0.2:0.8:5:3. The embedded epitope is AYKAAA which occupies positions 7 through 12 of the EERP. In the EERP, Ac indicates that the N-terminal amino acid is acetylated and amide indicates that the C-terminal amino acid of the amidated.

FIG. 2 is a graph showing prophylactic treatment of MOG-induced EAE in mice (an animal model for multiple sclerosis (MS)) using an EERP according to the present invention. EAE was induced in C57B1/6 female mice at 10 weeks of age by MOG₃₅₋₅₅ (Control; depicted as diamonds) and treated prophylactically by co-administration of Glatopa (depicted as triangles), PT-001 (YFAK random copolymer, depicted as open squares), or PT-002 (EERP consisting of linear random polypeptide YFAK with the peptide AYKAAA embedded in it; depicted as cross) at 500 μg/dose each. Mice (12 per treatment group) were monitored for signs of disease for 28 days post immunization. Numbers on the ordinate represent mean clinical score of clinical symptoms monitored for the days shown on the abscissa.

FIG. 3 is a graph showing prophylactic treatment of MOG-induced EAE in mice using two different doses of an EERP according to the present invention. EAE was induced in C57B1/6 female mice at 10 weeks of age by MOG₃₅₋₅₅ (Control, depicted as diamonds) and treated prophylactically by co-administration of FTY720 (fingolimod, depicted as triangles), PT-002 (EERP consisting of linear random polypeptide YFAK with the peptide AYKAAA embedded in it) at 150 μg/dose (depicted as open squares) at 150 μg/dose, or PT-002 at 500 μg/dose (depicted as cross). Mice (10 per treatment group) were monitored for signs of disease for 28 days post immunization. Numbers on the ordinate represent mean clinical score of clinical symptoms monitored for the days shown on the abscissa.

FIG. 4 is a graph showing prophylactic treatment of collagen-induced arthritis (CIA) in mice (animal model for rheumatoid arthritis (RA)) using an EERP according to the present invention. RA was induced in DBA/1 male mice at 8 weeks of age by collagen (Control, depicted as diamonds) and treated prophylactically by co-administration of PT-001 (YFAK random copolymer, depicted as triangles), or PT-002 (depicted as squares), each at 150 μg/dose. Mice (15 per treatment group) were monitored for signs of disease for 42 days post immunization. Numbers on the ordinate represent mean clinical score of clinical symptoms monitored for the days shown on the abscissa.

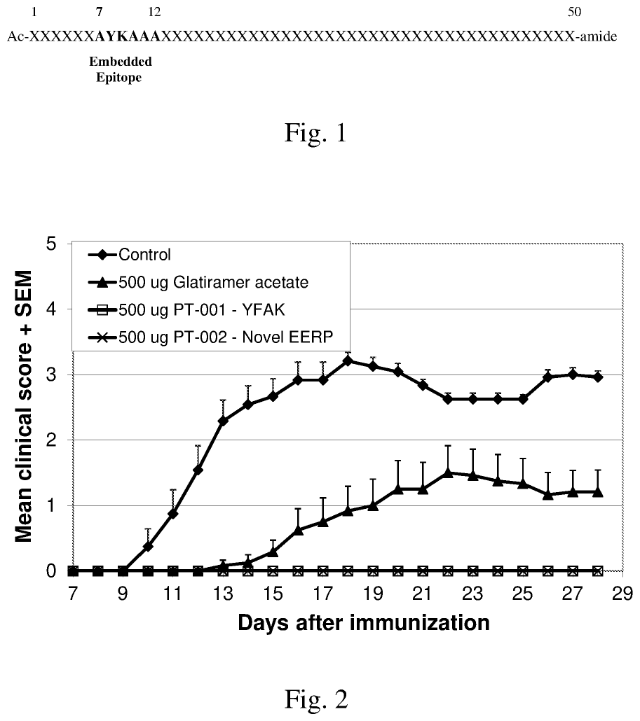

### DETAILED DESCRIPTION OF SPECIFIC EMBODIMENTS

### Definitions

Unless the context otherwise requires, as used in this description, the terms below shall have the meanings as set forth:

The term “autoimmune condition” means a disease state caused by an inappropriate immune response that is directed to a self-encoded entity, which is known as an autoantigen.

The term “immune-mediated condition” means a disease state mediated by an immune response that may have an autoimmune component but is not necessarily induced by the autoreactive immune compartment.

The term “anergy” means unresponsiveness of the immune system of a subject to an antigen.

The term “subject” means a mammal, preferably a human. The term “patient” refers to a human having an autoimmune disease.

The term EERP refers to Embedded Epitope Random Peptide. An EERP is a polypeptide consisting of two polypeptides: a first polypeptide consisting of a random sequence of three or more amino acids in a specific molar ratio, each amino acid occurring multiple times; and a second polypeptide defining an epitope composed of a specific amino acid sequence and embedded within the first polypeptide. An EERP has a fixed length and is capable of suppressing symptom and/or frequency of recurrent episodes of an autoimmune or immune-mediated disease.

The term “epitope,” as used herein, refers to a peptide sequence, assembled based on occupancy of certain anchor positions of an MHC class II molecule by amino acids of a peptide bound to the MHC molecule, preferably in the context of an autoimmune disease or condition. The epitope is not necessarily a peptide sequence found in a protein in the animal suffering from the autoimmune disease being treated by the copolymer containing the epitope. The peptide may originate from a bacteria or virus. Association between autoimmune disease and bacterial or viral infections are well known (see, for example, Sherbet, G. British Journal of Medical Practitioners, 2009, 2:(1), 6; Getts, D R et al. Immunol Rev. 2013, 255(1):197).

The term “derivative” of an amino acid means a chemically related form of that amino acid having an additional substituent, for example, N-carboxyanhydride group, a γ-benzyl group, an ε, N-trifluoroacetyl group, or a halide group attached to an atom of the amino acid.

The term “analog” means a chemically related form of that amino acid having a different configuration, for example, an isomer, or a D-configuration rather than an L-configuration, or an organic molecule with the approximate size, charge, and shape of the amino acid, or an amino acid with modification to the atoms that are involved in the peptide bond, so that the EERP having the analog residue is more protease resistant than an otherwise similar EERP lacking such analog, whether the analog is interior or is located at a terminus of the EERP, compared to the EERP without the analog.

The phrases “amino acid” and “EERP” can include one or more components which are amino acid derivatives and/or amino acid analogs as defined herein, the derivative or analog comprising part or the entirety of the residues for any one or more of the 20 naturally occurring amino acids indicated by that sequence. For example, in an EERP composition having one or more tyrosine residues, a portion of one or more of those residues can be substituted with homotyrosine. Further, an EERP having one or more non-peptide or peptidomimetic bonds between two adjacent residues is included within this definition.

The term “hydrophobic” amino acid means aliphatic amino acids alanine (A or ala), glycine (G or gly), isoleucine (I or ile), leucine (L or leu), proline (P or pro), and valine (V or val), the terms in parentheses being the one letter and three letter standard code abbreviations for each amino acid, and aromatic amino acids tryptophane (W or trp), phenylalanine (F or phe), and tyrosine (Y or tyr). These amino acids confer hydrophobicity as a function of the length of aliphatic and size of aromatic side chains, when found as residues within a copolymer or other polypeptide.

The term “charged” amino acid means amino acids aspartic acid (D or asp), glutamic acid (E or glu), histidine (H or his), arginine (R or arg) and lysine (K or lys), which confer a positive (his, lys and arg) or negative (asp, glu) charge at physiological values of pH on an aqueous solution of a EERP or other amino acid composition containing one or more residues of these amino acids.

The term “surface of class II MHC HLA-DR protein” includes the portions of the protein molecule in its 3-dimensional configuration which are in contact with its external environment, including those features of the protein that interact with aqueous solvent and are capable of binding to other cell components such as nucleic acids, other proteins, and peptides.

The term “antigen binding groove” refers to a 3-dimensional antigen interactive site on the surface of the class II MHC protein molecule that is formed by surfaces of both the α and β subunits of the class II MHC protein molecule.

The structure of the antigen binding groove of MHC class II HLA-DR or HLA-DQ proteins, each linked to a specific autoimmune condition or a number of conditions, is used as a basis herein to design EERP having compositions with potential therapeutic activity, as determined by binding to purified MHC class II proteins indicated above, in competition with a test compound which is a high affinity peptide. In some cases, a high affinity peptide could be an autoantigenic peptide, or Copaxone®.

Alternatively, the efficacy of the EERP could be tested by binding to living APC, for example, EBV-transformed B cell lines expressing a particular class II MHC protein associated with the autoimmune disease or a number of diseases. Furthermore, the therapeutic potential of the EERP is examined in antigen presentation assays using T cell hybridomas, T cell clones or whole blood from patients affected with the autoimmune condition.

The term “heterologous cell” means a cell for production of an MHC class II protein, which is unrelated to a cell of a subject, e.g., the heterologous cell is not a cell of a mammal. The heterologous cell for example can be from a cold-blooded animal, for example, from an invertebrate; the heterologous cell is an insect cell, or a cell of a microorganism such as a yeast cell. Following expression and production of MHC protein in a heterologous cell, the protein is free of any epitopes found in a mammal such as a human. Since the MHC protein is in an uncomplexed “empty” form, it is available for binding to the synthetic EERP of the present invention.

The terms “binding pockets” include 3-dimensional polymorphic regions of the peptide binding site of the class II MHC protein molecule that accommodates amino acid residue side chains from a peptide that is bound to the class II MHC protein including a bound naturally occurring antigen or epitope, and a bound synthetic peptide or EERP.

The term “substantially pure” as refers to a composition herein means that the material is primarily composed of the composition, and is largely free of other chemical materials. Purity can be assessed on the basis of weight, which can be determined by areas under a curve from a printout of an analytical instrument such as a gel reader, a chromatography column including gas chromatography, and other devices for purification known to those of skill in the biochemical arts.

The term “peptide” and “polypeptide” are used interchangeable and refers to two or more amino acids, amino acid derivatives, modified amino acids, and stereoisomers linked in a change.

The term “pharmaceutically acceptable carrier” includes any and all solvents, dispersion media, coatings, antimicrobials such as antibacterial and antifungal agents, isotonic and absorption delaying agents and the like that are physiologically compatible. Preferably, the carrier is suitable for intravenous, intramuscular, oral, rectal, intraperitoneal, transdermal, or subcutaneous administration, or administered as transdermal patch, and the active compound can be coated in a material to protect it from inactivation by the action of acids or other adverse natural conditions.

### Autoimmune Diseases

The immune system is able to recognize and generate reactions to a vast array of foreign materials, but not to self-antigens under regular circumstances, which leads to self-tolerance. Although recognition of self plays an important role in generating both the T cell and B cell repertoires of immune receptors and plays an essential role in the recognition of nominal antigen by T cells, the development of potentially harmful immune responses to self-antigens is, in general, precluded. Autoimmunity, therefore, represents the end result of the breakdown of one or more of the basic mechanisms regulating immune tolerance. The essential feature of an autoimmune disease is that tissue injury is caused by the immunologic reaction of the organism with its own tissues.

An autoimmune disease results from an inappropriate immune response directed against a self-antigen (an autoantigen), which is a deviation from the normal state of self-tolerance. Self-tolerance arises when the production of T cells and B cells capable of reacting against autoantigens has been prevented by events that occur in the development of the immune system during early life. The cell surface proteins that play a central role in regulation of immune responses through their ability to bind and present processed peptides to T cells are the MHC molecules (Rothbard J. B. et al., 1991. Annu. Rev. Immunol. 9:527).

An additional target for inhibition of an autoimmune response is the set of surface proteins called MHC molecules (HLA in humans). These molecules are located on chromosome 6 (6p21) and they help determine the individuality of mammalian tissues. The MHC region expresses a number of distinctive classes of MHC molecules in various cells of the body, the genes being, in order of sequence along the chromosome, the class I, II and III MHC genes. The class I gene region is subdivided into A, B and C subregions. The class II genes consist of DR, DQ and DP subregions, these gene products are expressed on the surface of APC such as macrophages, dendritic cells of lymphoid tissue and epidermal cells. The class III gene products are expressed in various components of the complement system, as well as in some non-immune related cells. Each of the MHC genes is found in a large number of alternative or allelic forms within a mammalian population. The genomes of subjects affected with certain autoimmune diseases are more likely to carry one or more characteristic MHC alleles, to which that disease is linked.

### MHC-Peptide Interactions

MHC molecules function as antigenic peptide receptors, and present bound peptides for T cell receptor (TCR) recognition. Detailed in vitro peptide binding and peptide elution studies and crystallographic data on MHC/peptide complexes over the past 25 years has led to enormous amount of information regarding the interaction of class II MHC molecules and their binding peptides ((Rammensee H. G. et al., 1995. Immunogenetics 41:178; Stern L. J. et al., 1994. Nature 368:215; Ghosh P. et al., 1995. Nature 378:457; Dessen A. et al., 1997. Immunity 7:473; Lee K. H. et al., 2001. Nat Immunol. 2:501). These studies have shown that the α and β chains of the class II MHC molecule orient themselves to form a 9 pocket peptide binding cleft. Pockets 1, 4, 6 and 9 are considered as the major peptide binding pockets while the other pockets, which are much shallower, are considered as minor pockets. Peptide-binding motifs for class II MHC molecules can be found on the following web sites: http://hlaligand.ouhsc.edu and http://syfpeithi.bmi-heidelberg.com/scripts/MHCServer.dL1/home.htm. Regarding the pathogenesis of autoimmune diseases, the elucidation of putative autoantigenic MHC binding epitopes in various autoimmune diseases provides an understanding of the specific MHC/peptide interactions that may be involved in disease development. The affinity of the interaction between MHC and peptides in critical, since it dictates the outcome of T cell engagement with the MHC/peptide complex.

### EERP

The EERP of the present invention comprises polypeptides consisting essentially of at least three amino acids combined in a specific molar ratio and polymerized into a random copolymer, having epitopes of specific sequence embedded into the random sequence. However, one of skill in the art can readily substitute structurally-related and/or charge-related amino acids without deviating from the spirit of the invention. Such conservative substitutions are structurally-related amino acid substitutions, including those amino acids which have about the same charge, hydrophobicity and size as the original EERP.

Moreover, the EERP can be composed of L- or D-amino acids. As is known by one skilled in the art, L-amino acids occur in most natural proteins. However, D-amino acids are commercially available and can be substituted for some or all of the amino acids used to make the biopolymers. The present invention contemplates EERP formed from mixtures of D- and L-amino acids, as well as EERP consisting of essentially L- or D-amino acids.

EERP of the present invention are capable of binding to MHC class II proteins. Any available method can be used to ascertain whether the EERP binds to one or more class II MHC proteins. For example, the EERP can be labeled with a reporter molecule (such as a radionucleotide or biotin), mixed with a crude or pure preparation of MHC class II protein and binding is detected if the reporter molecule adheres to the MHC class II protein after removal of the unbound polypeptide.

In some embodiments, EERP of the invention are capable of binding to an MHC class II protein associated with an arthritic condition, for example, RA or osteoarthritis. These EERP have a greater affinity for the antigen binding groove of an MHC class II protein associated with the autoimmune disease than does a type II collagen 261-273 peptide. Hence, these EERP can inhibit binding of or displace the type II collagen 261-273 peptide from the antigen-binding groove of an MHC class II protein. The class II MHC protein consists of approximately equal-sized α and β subunits, both of which are transmembrane proteins. A peptide-binding cleft is formed by parts of the amino termini of both a and β subunits. This peptide-binding cleft is the site of presentation of the antigen to T cells. There are at least three types of class II MHC molecules: HLA-DR, -DQ and -DP. There are also numerous alleles encoding each type of these HLA molecules. The class II MHC molecules are expressed predominantly on the surface of B lymphocytes and APC such as macrophages.

Amino acids are assembled into an EERP by peptide bonds in a random order with an embedded epitope of a specific sequence, using solid phase reaction or reaction in solution. In some embodiments, the EERP sequence includes at an amino acid modification or an amino acid that can inhibit proteolytic degradation of the EERP in a subject. For example, the amino acid proline can be included to inhibit proteolytic degradation. The proline can be present, for example, within four residues of at least one of the carboxy- and the amino-terminals. Also, one or more D-amino acids can be included in the EERP to inhibit proteolytic degradation. The D-amino acid can be present, for example, within four residues of at least one of the carboxy- and the amino-terminus. As for modification of an amino acid, the carboxy- or amino-terminus amino acid, or both may be modified. Further, the amino acid modification may be such that a non-peptide bond is introduced into the EERP. Exemplary non-peptide bonds include: a peptide nucleic acid bond, a methylene-amine bond, and a phosphorothioate bond. The amino acid modification can also be substitution of at least one alanine residue with a peptidomimetic compound, examples of which include: tetrahydroisoquinoline-(S)-3-carboxylic acid (Tic); tetrahydroisoquinoline-(S)-1-carboxylic acid (Thiq); dihydroisoindole-(S)-2-carboxylic acid (Disc); acetamido-methyl-Cys (C(Acm)); propylamidomethyl-Cys (C(Prm)); acetyl-Cys (C(Ace)); methylphenyl-Gly (MePhg); and norvaline (Nva). Further, the amino acid modification can be N-methylation of a peptide backbone nitrogen.

Another embodiment of the invention provides EERP with amino acid sequences capable of inhibiting the immune response to an autoantigen in a mammal, wherein the identity and position of at least one amino acid in the polypeptide sequence fits into at least one pocket of the peptide binding groove of an MHC class II protein.

### EERPs Composed of Y, F, A, and K (YFAK EERP)

In some embodiments, the first polypeptide of the EERP is composed of the amino acids Y, F, A, and K in a molar ratio of about 0.01-3.0:0.01-5.0:2-30:1-10, respectively.

In some related embodiments of the EERP, Y, F, A, and K, are present in a molar ratio of about respectively. In some other related embodiments of the EERP, Y, F, A, and K are present in a molar ratio of about 0.8-2.0:0.01-5.0:2-30:1-10, respectively. In some yet other related embodiments of the EERP, Y, F, A, and K are present in a molar ratio of about 0.5-1.5:0.01-5.0:2-30:1-10, respectively. In some preferred embodiments of the EERP, Y, F, A, and K are present in a molar ratio of about 0.2-1.0:0.01-5.0:2-30:1-10, respectively. In some more preferred embodiments of the EERP, Y, F, A, and K are present in a molar ratio of about 0.2-0.5:0.01-5.0:2-30:1-10, respectively. In some even more preferred embodiments of the EERP, Y, F, A, and K are present in a molar ratio of about 0.2:0.01-5.0:2-30:1-10, respectively.

In some related embodiments of the EERP, Y, F, A, and K are present in a molar ratio of about 0.01-3.0:0.1-5.0:2-30:1-10, respectively. In some other related embodiments of the EERP, Y, F, A, and K are present in a molar ratio of about 0.01-3.0:0.1-4.0:2-30:1-10, respectively. In some yet other related embodiments of the EERP, Y, F, A, and K are present in a molar ratio of about 0.01-3.0:0.8-3.0:2-30:1-10, respectively. In some preferred embodiments of the EERP, Y, F, A, and K are present in a molar ratio of about 0.01-3.0:0.8-2.0:2-30:1-10, respectively. In some more preferred embodiments of the EERP, Y, F, A, and K are present in a molar ratio of about 0.01-3.0:0.6-1.0:2-30:1-10, respectively. In some even more preferred embodiments of the EERP, Y, F, A, and K are present in a molar ratio of about 0.01-3.0:0.5:2-30:1-10, respectively.

In some related embodiments of the EERP, Y, F, A, and K are present in a molar ratio of about 0.01-3.0:0.1-5.0:2-25:1-10, respectively. In some other related embodiments of the EERP, Y, F, A, and K are present in a molar ratio of about 0.01-3.0:0.01-5.0:2-20:1-10, respectively. In some yet other embodiments of the EERP, Y, F, A, and K are present in a molar ratio of about 0.01-3.0:0.01-5.0:2-15:1-10, respectively. In some preferred embodiments of the EERP, Y, F, A, and K are present in a molar ratio of about 0.01-3.0:0.01-5.0:2-10:1-10, respectively. In some more preferred embodiments of the EERP, Y, F, A, and K are present in a molar ratio of about 0.01-3.0:0.01-5.0:5-10:1-10, respectively. In some even more preferred embodiments of the EERP, Y, F, A, and K are present in a molar ratio of about 0.01-3 .0: 0.01-5 .0: 5: 1-10, respectively.

In some related embodiments of the EERP, Y, F, A, and K are present in a molar ratio of about 0.01-3.0:0.01-5.0:2-30:2-8, respectively. In some other embodiments of the EERP, Y, F, A, and K are present in a molar ratio of about 0.01-3.0:0.01-5.0:2-30:2-6, respectively. In some yet other embodiments of the EERP, Y, F, A, and K are present in a molar ratio of about 0.01-3.0:0.01-5.0:2-30:2-5, respectively. In some preferred embodiments of the EERP, Y, F, A, and K are present in a molar ratio of about 0.01-3.0:0.01-5.0:2-30:2-4, respectively. In some more preferred embodiments of the EERP, Y, F, A, and K are present in a molar ratio of about 0.01-3.0:0.01-5.0:2-30:3-4, respectively. In some even more preferred embodiments of the EERP, Y, F, A, and K are present in a molar ratio of about 0.01-3.0:0.01-5.0:2-30:3, respectively.

In some embodiments of the EERP, Y, F, A, and K are present in a molar ratio of about 0.1-2.5:0.1-5.0:2-25:2-8, respectively. In some embodiments of the EERP, Y, F, A, and K are present in a molar ratio of about 0.8-2.0:0.1-4.0:2-20:2-6, respectively. In some embodiments of the EERP, Y, F, A, and K are present in a molar ratio of about 0.5-1.5:0.8-3.0:2-15:2-5, respectively. In some embodiments of the EERP, Y, F, A, and K are present in a molar ratio of about 0.2-1.0:0.8-2.0:2-10:2-4, respectively. In some more preferred embodiments of the EERP, Y, F, A, and K are present in a molar ratio of about respectively. In some even more preferred embodiments of the EERP, Y, F A, and K are present in a molar ratio of about 0.2:0.5:5:3, 0.2:0.8:5:3, 0.5:0.5:5:3, or 0.8:0.2:5:3, respectively.

### Other EERP

Contemplated within the invention, and as described below, are embodiments, in in which the first polypeptide of the EERP is a linear random amino acid copolymer composed of a combination of amino acids other that Y, F, A, and K. These random amino acid polymer component of the EERP have been previously described (see, for example, Stern et al., PNAS 2004, 101(32): 11743; Fridkis-Hareli et el., J. Clin. Invest. 2002, 109(12): 1635; Fridkis-Hareli et al., PNAS 1998, 95(21):12528; Fridkis-Hareli et al. J Immunol. 1999, 162(8):4697; Fridkis-Hareli et al., Hum Immunol 2000, 61(7): 640; Fridkis-Hareli et al., Hum Immunol 2001, 62(8): 753; and WO 2003/029276).

In some embodiments, the first polypeptide of the EERP is a linear random amino acid copolymer composed of valine (V), phenylalanine (F), alanine (A) and lysine (K) (VFAK EERP). In these EERP, V, F, A, and K may be present in a molar ratio of about 0.01-3:0.01-5.0:2-30:1-10 respectively. In some related embodiments, V, F, A, and K are present in a molar ratio of about 0.1-2.5:0.1-5.0:2-25:2-8, respectively. In some other related embodiments, V, F, A, and K are present in a molar ratio of about 0.8-2.0:0.1-4.0:2-20:2-6, respectively. In some yet other related embodiments, V, F, A, and K are present in a molar ratio of about 0.5-1.5:0.8-3.0:2-15:2-5, respectively. In some preferred embodiments, V, F, A, and K are present in a molar ratio of about 0.2-1.0:0.8-2.0:2-10:2-4, respectively. In some more preferred embodiments, V, F, A, and K are present in a molar ratio of about 0.2-0.5:0.6-1.0:5-10:3-4, respectively. In some even more preferred embodiments, V, F, A, and K are present in a molar ratio of about 0.2:0.5:5:3, 0.2:0.8:5:3, 0.5:0.5:5:3, or 0.8:0.2:5:3, respectively.

In some embodiments, the first polypeptide of the EERP is a linear random amino acid copolymer composed of valine (V), tryptophan (W), alanine (A) and lysine (K) (VWAK EERPs). In these EERP, V, W, A, and K may be present in a molar ratio of about 0.01-3:0.01-5.0:2-30:1-10 respectively. In some related embodiments, V, W, A, and K are present in a molar ratio of about 0.1-2.5:0.1-5.0:2-25:2-8, respectively. In some other related embodiments, V, W, A, and K are present in a molar ratio of about 0.8-2.0:0.1-4.0:2-20:2-6, respectively. In some yet other embodiments, V, W, A, and K are present in a molar ratio of about 0.5-1.5:0.8-3.0:2-15:2-5, respectively. In some preferred embodiments, V, W, A, and K are present in a molar ratio of about 0.2-1.0:0.8-2.0:2-10:2-4, respectively. In some more preferred embodiments, V, W, A, and K are present in a molar ratio of about 0.2-0.5:0.6-1.0:5-10:3-4, respectively. In some even more preferred embodiments, V, W, A, and K are present in a molar ratio of about 0.2:0.5:5:3, 0.2:0.8:5:3, 0.5:0.5:5:3, or 0.8:0.2:5:3, respectively.

In some embodiments, the first polypeptide of the EERP is a linear random amino acid copolymer composed of valine (V), tyrosine (Y), alanine (A) and lysine (K) (VYAK EERP). In these EERP, V, Y, A, and K may be present in a molar ratio of about 0.01-3:0.01-5.0:2-30:1-10 respectively. In some related embodiments, V, Y, A, and K are present in a molar ratio of about 0.1-2.5:0.1-5.0:2-25:2-8, respectively. In some other related embodiments, V, Y, A, and K are present in a molar ratio of about 0.8-2.0:0.1-4.0:2-20:2-6, respectively. In some yet other embodiments, V, Y, A, and K are present in a molar ratio of about 0.5-1.5:0.8-3.0:2-15:2-5, respectively. In some preferred embodiments, V, Y, A, and K are present in a molar ratio of about 0.2-1.0:0.8-2.0:2-10:2-4, respectively. In some more preferred embodiments, V, Y, A, and K are present in a molar ratio of about 0.2-0.5:0.6-1.0:5-10:3-4, respectively. In some even more preferred embodiments, V, Y, A, and K are present in a molar ratio of about 0.2:0.5:5:3, 0.2:0.8:5:3, 0.5:0.5:5:3, or 0.8:0.2:5:3, respectively.

In some embodiments, the first polypeptide of the EERP is a linear random amino acid copolymer composed of glutamic acid (E), tyrosine (Y) alanine (A), and lysine (K) (EYAK EERP). It should be noted that this random amino acid copolymer is same as Copaxone® (Cop 1; Teitelbaum D. et al. 1971. Eur. J. Immunol. 1:242-248) when A, K, E, and Y are present in a molar ratio of approximately Cop 1 is a widely used therapy for MS. Cop 1 reduces the MS relapse rate, but does not eliminate relapse or cure the disease (Bornstein, M. B. et al. 1987. N. Engl. J. Med. 317:408-414; Johnson, K. P. et al. 1995. Neurology 45:1268-1276; Johnson, K. P. et al. 1998. Neurology 50:701-708). In these EERP, E, Y, A, and K may be present in a molar ratio of about 0.01-3:0.01-5.0:2-30:1-10 respectively. In some related embodiments, E, Y, A, and K are present in a molar ratio of about 0.1-2.5:0.1-5.0:2-25:2-8, respectively. In some other related embodiments, E, Y, A, and K are present in a molar ratio of about 0.8-2.0:0.1-4.0:2-20:2-6, respectively. In some yet other related embodiments, E, Y, A, and K are present in a molar ratio of about 0.5-1.5:0.8-3.0:2-15:2-5, respectively. In some preferred embodiments, E, Y, A, and K are present in a molar ratio of about 0.2-1.0:0.8-2.0:2-10:2-4, respectively. In some more preferred embodiments, E, Y, A, and K are present in a molar ratio of about 0.2-0.5:0.6-1.0:5-10:3-4, respectively. In some even more preferred embodiments, E, Y, A, and K are present in a molar ratio of about 0.2:0.5:5:3, 0.2:0.8:5:3, 0.5:0.5:5:3, or 0.8:0.2:5:3, respectively.

In some embodiments, the first polypeptide of the EERP is a linear random amino acid copolymer composed of phenylalanine (F), alanine (A) and lysine (K) (FAK EERPs). In these EERP, F, A, and K may be present in a molar ratio of 0.01-5.0:2-30:1-10 respectively. In some related embodiments, F, A, and K are present in a molar ratio of about 0.1-5.0:2-25:2-8, respectively. In some other related embodiments, F, A, and K are present in a molar ratio of about 0.1-4.0:2-20:2-6, respectively. In some yet other related embodiments, F, A, and K are present in a molar ratio of about 0.8-3.0:2-15:2-5, respectively. In some preferred embodiments, F, A, and K are present in a molar ratio of about 0.8-2.0:2-10:2-4, respectively. In some more preferred embodiments, F, A, and K are present in a molar ratio of about 0.6-1.0:5-10:3-4, respectively. In some even more preferred embodiments, F, A, and K are present in a molar ratio of about 0.5:5:3, 0.8:5:3, or 1:5:3, respectively.

In some embodiments, the first polypeptide of the EERP is a linear random amino acid copolymer composed of valine (V), alanine (A) and lysine (K) (VAK EERP). In these EERP, V, A, and K may be present in a molar ratio of 0.01-5.0:2-30:1-10 respectively. In some related embodiments, V, A, and K are present in a molar ratio of about 0.1-5.0:2-25:2-8, respectively. In some other related embodiments, V, A, and K are present in a molar ratio of about 0.1-4.0:2-20:2-6, respectively. In some yet other related embodiments, V, A, and K are present in a molar ratio of about 0.8-3.0:2-15:2-5, respectively. In some preferred embodiments, V, A, and K are present in a molar ratio of about 0.8-2.0:2-10:2-4, respectively. In some more preferred embodiments, V, A, and K are present in a molar ratio of about 0.6-1.0:5-10:3-4, respectively. In some even more preferred embodiments, V, A, and K are present in a molar ratio of about 0.5:5:3, 0.8:5:3, or 1:5:3, respectively.

In some embodiments, the first polypeptide of the EERP is a linear random amino acid copolymer composed of tryptophan (W), alanine (A) and lysine (K) (WAK EERPs). In these EERP, W, A, and K may be present in a molar ratio of 0.01-5.0:2-30:1-10 respectively. In some related embodiments, W, A, and K are present in a molar ratio of about 0.1-5.0:2-25:2-8, respectively. In some other related embodiments, W, A, and K are present in a molar ratio of about 0.1-4.0:2-20:2-6, respectively. In some yet other related embodiments, W, A, and K are present in a molar ratio of about respectively. In some preferred embodiments, W, A, and K are present in a molar ratio of about 0.8-2.0:2-10:2-4, respectively. In some more preferred embodiments, W, A, and K are present in a molar ratio of about 0.6-1.0:5-10:3-4, respectively. In some even more preferred embodiments, W, A, and K are present in a molar ratio of about 0.5:5:3, 0.8:5:3, or 1:5:3, respectively.

In some embodiments, the first polypeptide of the EERP is a linear random amino acid copolymer composed of tyrosine (Y), alanine (A) and lysine (K) (YAK EERPs). In these EERP, Y, A, and K may be present in a molar ratio of 0.01-5.0:2-30:1-10 respectively. In some related embodiments, Y, A, and K are present in a molar ratio of about 0.1-5.0:2-25:2-8, respectively. In some other related embodiments, Y, A, and K are present in a molar ratio of about 0.1-4.0:2-20:2-6, respectively. In some yet other related embodiments, Y, A, and K are present in a molar ratio of about 0.8-3.0:2-15:2-5, respectively. In some preferred embodiments, Y, A, and K are present in a molar ratio of about 0.8-2.0:2-10:2-4, respectively. In some more preferred embodiments, Y, A, and K are present in a molar ratio of about 0.6-1.0:5-10:3-4, respectively. In some even more preferred embodiments, Y, A, and K are present in a molar ratio of about 0.5:5:3, 0.8:5:3, or 1:5:3, respectively.

In some embodiments, the first polypeptide of the EERP is a linear random amino acid copolymer composed of tryptophan (Y), glutamic acid (E) and lysine (K) (YEK EERPs). In these EERP, Y, E, and K may be present in a molar ratio of 0.01-5.0:2-30:1-10 respectively. In some related embodiments, Y, E, and K are present in a molar ratio of about 0.1-5.0:2-25:2-8, respectively. In some other related embodiments, Y, E, and K are present in a molar ratio of about 0.1-4.0:2-20:2-6, respectively. In some yet other related embodiments, Y, E, and K are present in a molar ratio of about respectively. In some preferred embodiments, Y, E, and K are present in a molar ratio of about 0.8-2.0:2-10:2-4, respectively. In some more preferred embodiments, Y, E, and K are present in a molar ratio of about 0.6-1.0:5-10:3-4, respectively. In some even more preferred embodiments, Y, E, and K are present in a molar ratio of about 0.5:5:3, 0.8:5:3, or 1:5:3, respectively.

In some embodiments, the first polypeptide of the EERP is a linear random amino acid copolymer composed of tryptophan (Y), glutamic acid (E) and alanine (A) (YEA EERPs). In these EERP, Y, E, and A may be present in a molar ratio of 0.01-5.0:2-30:1-10 respectively. In some related embodiments, Y, E, and A are present in a molar ratio of about 0.1-5.0:2-25:2-8, respectively. In some other related embodiments, Y, E, and A are present in a molar ratio of about 0.1-4.0:2-20:2-6, respectively. In some yet other related embodiments, Y, E, and A are present in a molar ratio of about respectively. In some preferred embodiments, Y, E, and A are present in a molar ratio of about 0.8-2.0:2-10:2-4, respectively. In some more preferred embodiments, Y, E, and A are present in a molar ratio of about 0.6-1.0:5-10:3-4, respectively. In some even more preferred embodiments, Y, E, and A are present in a molar ratio of about 0.5:5:3, 0.8:5:3, or 1:5:3, respectively.

In some embodiments, the first polypeptide of the EERP is a linear random amino acid copolymer VEAK comprising valine (V), glutamic acid (E), alanine (A) and lysine (K) (VEAK EERPs). In these EERP, V, E, A, and K may be present in a molar ratio of about 0.01-3:0.01-5.0:2-30:1-10 respectively. In some related embodiments, V, E, A, and K are present in a molar ratio of about 0.1-2.5:0.1-5.0:2-25:2-8, respectively. In some other related embodiments, V, E, A, and K are present in a molar ratio of about 0.8-2.0:0.1-4.0:2-20:2-6, respectively. In some yet other embodiments, V, E, A, and K are present in a molar ratio of about 0.5-1.5:0.8-3.0:2-15:2-5, respectively. In some preferred embodiments, V, E, A, and K are present in a molar ratio of about 0.2-1.0:0.8-2.0:2-10:2-4, respectively. In some more preferred embodiments, V, E, A, and K are present in a molar ratio of about 0.2-0.5:0.6-1.0:5-10:3-4, respectively. In some even more preferred embodiments, V, E, A, and K are present in a molar ratio of about 0.2:0.5:5:3, 0.2:0.8:5:3, 0.5:0.5:5:3, 0.8:0.2:5:3, or 1:1.5:5:3, respectively.

In some embodiments, the first polypeptide of the EERP is a linear random amino acid copolymer comprising phenylalanine (F), glutamic acid (E), alanine (A) and lysine (K) (FEAK EERPs). In these EERP, F, E, A, and K may be present in a molar ratio of about 0.01-3:0.01-5.0:2-30:1-10 respectively. In some related embodiments, F, E, A, and K are present in a molar ratio of about 0.1-2.5:0.1-5.0:2-25:2-8, respectively. In some other related embodiments, F, E, A, and K are present in a molar ratio of about 0.8-2.0:0.1-4.0:2-20:2-6, respectively. In some yet other embodiments, F, E, A, and K are present in a molar ratio of about 0.5-1.5:0.8-3.0:2-15:2-5, respectively. In some preferred embodiments, F, E, A, and K are present in a molar ratio of about 0.2-1.0:0.8-2.0:2-10:2-4, respectively. In some more preferred embodiments, F, E, A, and K are present in a molar ratio of about 0.2-0.5:0.6-1.0:5-10:3-4, respectively. In some even more preferred embodiments, F, E, A, and K are present in a molar ratio of about 0.2:0.5:5:3, 0.2:0.8:5:3, 0.5:0.5:5:3, 0.8:0.2:5:3, or 1:1.5:5:3, respectively.

Further, any of the EERP provided here may be provided in a composition that in addition includes pharmaceutically acceptable buffer, and/or in a unit dosage.

The EERP herein are comprised of amino acids as described, and, with regard to the first polypeptide of any given EERP, are further considered to be equivalent to copolymers sharing the amino acid compositions as described and also containing one or more additional substituents, for example, have one or more additional amino acids, such that the resulting copolymer has about the same function. For example, a copolymer FEAK, FAK, VWAK, VEAK, YFAK, or any of the copolymer compositions as provided herein, which is comprised substantially of this composition, i.e, is at least about 60%, or at least about 70%, or at least about 80%, or at least about 90%, or at least about 95% or about 99% the composition provided herein, and has about the same functional properties as a copolymer provided herein, is considered equivalent to the composition as provided herein. The function is considered to be about the same if a dosage of a composition herein comprising the EERP that is effective for treating an autoimmune disease is about the same as a dosage of a copolymer comprising substantially the same substituents as a composition herein, for treating the autoimmune disease

### Pharmaceutical Carriers Contemplated by the Invention

The present EERP can be formulated into pharmaceutical compositions containing a pharmaceutically acceptable carrier. As used herein, “pharmaceutically acceptable carrier” includes any and all solvents, dispersion media, coatings, antibacterial and antifungal agents, isotonic and absorption delaying agents, sweeteners and the like. The pharmaceutically acceptable carriers may be prepared from a wide range of materials including, but not limited to, flavoring agents, sweetening agents and miscellaneous materials such as buffers and absorbents that may be needed in order to prepare a particular therapeutic composition. The use of such media and agents with pharmaceutically active substances is well known in the art. Except insofar as any conventional media or agent is incompatible with the active ingredient, its use in the therapeutic compositions is contemplated. Supplementary active ingredients can also be incorporated into the compositions. The present compositions may be formulated as an injectable solution or suspension, a spray solution or a suspension.

Preferably, the carrier is suitable for intravenous, intramuscular, oral, intraperitoneal, transdermal, or subcutaneous administration, and the active compound can be coated in a material to protect it from inactivation by the action of acids or other adverse natural conditions. The methods of the invention include incorporation of an EERP into a pharmaceutical composition suitable for administration to a subject. A composition of the present invention can be administered by a variety of methods known in the art as will be appreciated by the skilled artisan. The active compound can be prepared with carriers that will protect it against rapid release formulation, including implants, transdermal patches, and microencapsulated delivery systems. Many methods for the preparation of such formulations are patented and are generally known to those skilled in the art. (See, e.g., Sustained and Controlled Release Drug Delivery systems, J. R. Robinson, Ed., Marcel Dekker, Inc., NY, 1978). Therapeutic compositions for delivery in a pharmaceutically acceptable carrier are sterile, and are preferably stable under the conditions of manufacture and storage. The composition can be formulated as a solution, microemulsion, liposome, or other ordered structure suitable to high drug concentration.

Examples of carriers include colloidal drug carrier systems, which are poly(hydroxyethylaspartamide) derivatives (PHEA) bearing at the polyaminoacidic backbone poly(ethyleneglycol) (2,000 or 5,000 Da) or both poly(ethyleneglycol) and hexadecylalkylamine. Microparticles, nanoparticles and liposomes are additional examples of colloidal carriers. Lactose is an example for carrier for dry powder inhalers (DPIs). Other carrier systems are bioinspired, pH-responsive polymeric carriers, which target and direct cellular uptake, as well as enhance cytosolic delivery by disrupting endosomal membranes in a pH-dependent fashion; bioadhesive microspheres polymers target drugs to specific cells and intracellular compartments; polymer micelles providing protection of the drug from biological degradation and denaturation; immunomicelles in which antibodies are chemically attached are used for solubilization of poorly soluble drugs; non-ionic surfactant vesicles for receptor-based drug delivery; cyclodextrins as sustained-release carriers; biodegradable polymers as carriers of pharmaceutical compounds. Long circulating pharmaceutical carriers prolong the circulation half-life of the microparticulate systems.

### EERP Size and Composition Contemplated by the Invention

In one embodiment, the EERP is a mixture of linear random polypeptides of about 30 to 200 amino acids with embedded epitopes of about 5 to 20 amino acids in length. For example, the EERP is composed of linear random polypeptides of about 50 amino acids in length; for example, the embedded epitope is 6 amino acids in length.

In a related embodiment, a linear random polypeptide comprises of amino acids Y, F, A and K; a nested peptide is a sequence of amino acids AYKAAA that starts at position 7 from the N-terminus of the random polypeptide.

### Therapeutic Compositions and Methods Contemplated by the Invention

The present invention further provides methods and compositions for treating and preventing autoimmune diseases in a mammal which include administering a therapeutically effective amount of a composition having a polypeptide containing at least 3 different amino acids, wherein the selected amino acids are randomly polymerized in a linear configuration, while other selected amino acids are placed at fixed positions and polymerized in a linear configuration.

Autoimmune diseases contemplated by the present invention include either cell-mediated diseases (e.g. T-cell) or antibody-mediated (e.g. B cell) disorders. The present compositions can be used to treat one or more of these diseases.

In one embodiment, any autoimmune disease can be treated by the present EERP so long as the contemplated EERP binds to an MHC class II protein that has been associated with the autoimmune disease. One aspect of this embodiment provides a method for developing an EERP or composition which includes selecting a EERP that inhibits binding of an antigenic peptide to class II MHC protein, for example, a method wherein step (a) further comprises selecting the EERP that inhibits class II-specific T cell responses to an MHC class II protein-peptide complex, and a method wherein the antigenic peptide is associated with an autoimmune disease; in another embodiment of the invention, a method is provided wherein the MHC class II protein is associated with an autoimmune disease.

In another embodiment, the method or composition for treating an autoimmune disease in a mammal further involves inhibiting the proliferation or function of T cells which are responsive to an autoantigen. The pathological process of autoimmune diseases and immune rejection is mediated by T cells. Upon binding to and recognition of an antigen, T cells proliferate, secrete cytokines and recruit additional inflammatory and cytotoxic cells to the site. The present polypeptides prevent T cell proliferation and T cell functions such as cytokine secretion and recruitment of inflammatory and cytotoxic cells to the site. When the autoimmune disease is an arthritic condition, the autoantigen can be collagen, and the present polypeptides can inhibit the proliferation and function of collagen-responsive T cells.

In another embodiment, the method or composition for treating an autoimmune disease in a mammal involves binding the polypeptide to an antigen presenting cell such as a macrophage, a dendritic cell of the lymphoid tissue or an epidermal cell. The proliferation and functions of a T cell are activated when an appropriate antigen is presented to it. By binding to APC, the present polypeptides may block or otherwise interfere with T cell activation.

In yet another embodiment, the method or composition for treating an autoimmune disease in a mammal involves binding the polypeptide to MHC class II protein which is associated with an autoimmune disease. The class II MHC proteins are expressed predominantly on the surface of B lymphocytes and APC such as macrophages. These class II MHC proteins have peptide-binding cleft which is the site at which antigenic peptides are presented to T cells. When the present EERP bind to MHC class II protein, they can block or otherwise interfere with antigen presentation and/or T cell activation.

In another embodiment, the method or composition for treating an autoimmune disease in a mammal involves binding of EERP to specific B cell antibodies, and/or specific T cells. When binding to specific T cells, the EERP of the present invention stimulates those T cells to proliferate, secrete anti-inflammatory cytokines and enhance the therapeutic benefits of treatment by the present methods. According to the present invention, the EERP also bind to autoantigen-reactive antibodies which may block the antibody from attacking the target tissue, thereby helping to prevent the autoimmune disease from progressing. For example, when the EERP are bound to MBP-specific antibodies, those antibodies may not bind to MBP and therefore prevent the destruction of MBP in the myelin sheath.

The EERP may be administered by any conventional route. In one embodiment the EERP can be administered by injection to facilitate delivery to the tissues affected by the autoimmune disease. Thus, the EERP may, for example, be injected, ingested, inhaled, or topically applied. The EERP may be incorporated into a cream, solution or suspension for topical administration. The EERP are preferably administered orally, topically or by injection without addition of an adjuvant.

### Useful Kits of the Invention

Another embodiment of the invention provides a kit for assaying the binding of an analyte to an MHC protein, which includes a water-soluble MHC protein, which has been recombinantly produced in a non-mammalian cell, and a means for detection of the bound analyte on the MHC protein, and instruction for use. The MHC protein used in the kit is an MHC class II protein selected from the group consisting of an MHC class II HLA-DR1 protein, an MHC class II HLA-DQ2 protein, an MHC class II HLA-DQ8 protein, and an MHC class II DR4 protein. The kit further comprises an autoantigenic peptide. The kit further comprises a high affinity binding peptide.

In a preferred embodiment, the MHC class II protein is produced in an invertebrate or a microbial cell, such as an insect cell or a yeast cell and is therefore devoid of bound peptide in the antigen cleft. The means for detecting binding of the analyte to the MHC protein can be any radioactive, fluorometric, chemiluminescent, enzymatic or colorimetric means known to one skilled in the art. In a preferred embodiment, the MHC protein is selected from class II HLA-DR1, DR4, DQ2, DQ8 or a different allele associated with the immune-mediated condition. Further, the kit can include also an autoantigenic peptide, such as collagen type II peptide, or a peptide from some other protein implicated in an autoimmune disease. Furthermore, the kit can include measuring T cell responses, B cell responses, cytokines, chemokines by methods known to one skilled in the art.

### Manufacturing and Testing of the EERP of the Invention

The EERP can be made by any procedure available to one skilled in the art. For example, EERP of the present invention are synthesized by the solid-phase method using Fmoc amino acids mixed in the desired ratios at each cycle. EERP is a mixture of linear random polypeptides composed of amino acids Y, F, A and K at the molar ratios of 0.2:0.8:5:3, respectively. The nested epitope consists of the amino acid sequence AYKAAA starting at position 7 from the N-terminus of the polypeptide. The average length of the EERP is 52 amino acids.

The EERP of the invention is further tested in vivo in a prophylactic model of MS, EAE, which is induced in female mice of C57B1/6J strain by MOG 35-55 encephalitogenic peptide co-administered with the EERP in Complete Freund's Adjuvant. Each group consists of 12 mice. Four experimental groups are MOG 35-55 only, Glatopa+MOG 35-55, random copolymer YFAK+MOG 35-55 and EERP+MOG 35-55 at 500 μg per single dose each. Disease scoring begins on Day 7 and is performed daily till Day 28. Disease severity is based on clinical observations and is assessed on the scale from 0 to 5. The incidence of EAE was 100% in the MOG 35-55 control group, 67% in the Glatopa treated group and 0% in the PT-001 (YFAK) and in PT-002 (EERP) treated groups.

It is to be understood and expected that variations in the principles of invention herein disclosed may be made by one skilled in the art and it is intended that such modifications are to be included within the scope of the present invention.

While the invention has been described and illustrated herein by references to various specific materials, procedures, and examples, it is understood that the invention is not restricted to the particular combination of materials and procedures selected for that purpose. Numerous variations can be implied as will be appreciated by those skilled in the art. It is intended that the specification and examples be considered exemplary, only, with the true scope and spirit of the invention being indicated by the following claims. All references, patents, and patent applications referred to in this application are hereby incorporated by reference in their entirety.

### EXAMPLES

### Materials and Methods

Peptide synthesis. PT-001 which is a random copolymer of the amino acids YFAK at a ratio of was manufactured by solid phase synthesis as a 50-mer on pre-loaded Wang resin with base labile Fmoc-groups (Vivitide, Gardner, MA). The excess amino acid derivatives and coupling reagents were removed by filtration. EERP was cleaved, N-acetylated (depicted as Ac), C-amidated (depicted as amide), HPLC purified, precipitated, washed and dried under vacuum. The sequence of YFAK is Ac-XXXXXXXXXXXXXXXXXXXXXXXXXXXXXXXXXXXXXXXXXXXXXXXXXX-amide where X is a random sequence of the amino acids YFAK.

PT-002 was manufactured by solid phase synthesis as a 52-mer on pre-loaded Wang resin with base labile Fmoc-groups (Vivitide, Gardner, MA). The sequence of PT-002 is Ac-XXXXXXAYKAAAXXXXXXXXXXXXXXXXXXXXXXXXXXXXXXXXXXXXXXXX-amide where X designates a random sequence of YFAK at a ratio of 0.2:0.8:5:3. The embedded epitope is of the sequence AYKAAA and begins at position 7. The excess amino acid derivatives and coupling reagents were removed by filtration. EERP was cleaved, N-acetylated (depicted as Ac), C-amidated (depicted as amide), HPLC purified, precipitated, washed and dried under vacuum.

Glatopa (glatiramer acetate for injection) at 20 mg/mL in pre-filled syringes was obtained from Mika Pharmaceuticals (Plainview, NY).

### Example 1

Cell-based assays. Human monocytic cell line THP-1 (ATCC, Manassas, VA) was cultured in RPMI media supplemented with 10% fetal bovine serum (FBS) and antibiotics penicillin/streptomycin in a 37° C. 5% CO 2 incubator. Cells were plated at 0.25×10⁶ cells/well in a 24-well cell culture plate and pre-incubated for 1.5 hr with Glatopa, PT-001 or PT-002 at the final concentration 0.1 μg/mL or 10 μg/mL each, followed by addition of lipopolysaccharide (LPS) at the final concentration of 1 μg/mL and incubation for 24 hr in a 37° C. 5% CO₂ incubator. Plates were centrifuged and supernatants (100 μL) were tested for levels of pro-inflammatory cytokines IL-1β, IL-6 and TNF-α using commercial ELISA kits (R&D System for IL-1β, BioLegend for IL-6 and TNF-α). The data in Table 1 show higher levels of inhibition of LPS-induced secretion of pro-inflammatory cytokines in presence of PT-002 as compared to PT-001 or Glatopa. Percent inhibition is calculated as 100-(cytokine concentration in presence of inhibitor/cytokine concentration without inhibitor*100).

TABLE 1
Inhibition of pro-inflammatory cytokine secretion
by LPS-stimulated human monocytic cell line THP-1
in presence of Glatopa, PT-001/YFAK and PT-002/EERP

| | Final Concentration | Percent inhibition (%) | | |
|---|---|---|---|---|
| Compound | (μg/mL) | IL-1b | IL-6 | TNF-a |
| Glatopa | 0.1 | 22 | −10 | −162 |
| | 10 | 42 | −21 | −37 |
| PT-001/YFAK | 0.1 | 21 | −8 | −5 |
| | 10 | 77 | 84 | 34 |
| PT-002/EERP | 0.1 | 53 | 31 | 28 |
| | 10 | 99 | 100 | 96 |

### Example 2

Prophylactic treatment of EAE, a mouse model of MS. C57B1/6 female mice (Taconic Biosciences) were acclimated for 2 weeks prior to the start of the study. As shown in FIG. 2, EAE was induced in mice at 10 weeks of age by MOG₃₅₋₅₅ (Control, depicted as diamonds) and treated prophylactically by co-administration of a single dose in immunization emulsion containing MOG ₃₅₋₅₅ and Glatopa (depicted as triangles), MOG₃₅₋₅₅ and PT-001 (depicted as open squares), or MOG₃₅₋₅₅ and PT-002 (depicted as cross) at 500 μg/dose each. All compounds were emulsified with CFA and MOG₃₅₋₅₅ and used to immunize mice. Injections were subcutaneous at 2 sites in the back (upper and lower) at the volume of 0.1 mL at each site. Within 2 hours of the injection of emulsion, and again 24 hr after the injection of emulsion, pertussis toxin was administered intraperitoneally. Mice (12 per treatment group) were monitored daily in a blind fashion starting from Day 7 for signs of disease for 28 days post immunization. In contrast to the Control group or Glatopa-treated group, mice administered with either PT-001 or PT-002 did not exhibit any signs of disease for the duration of the study. Study was performed by Hooke Labs (Lawrence, MA).

### Example 3

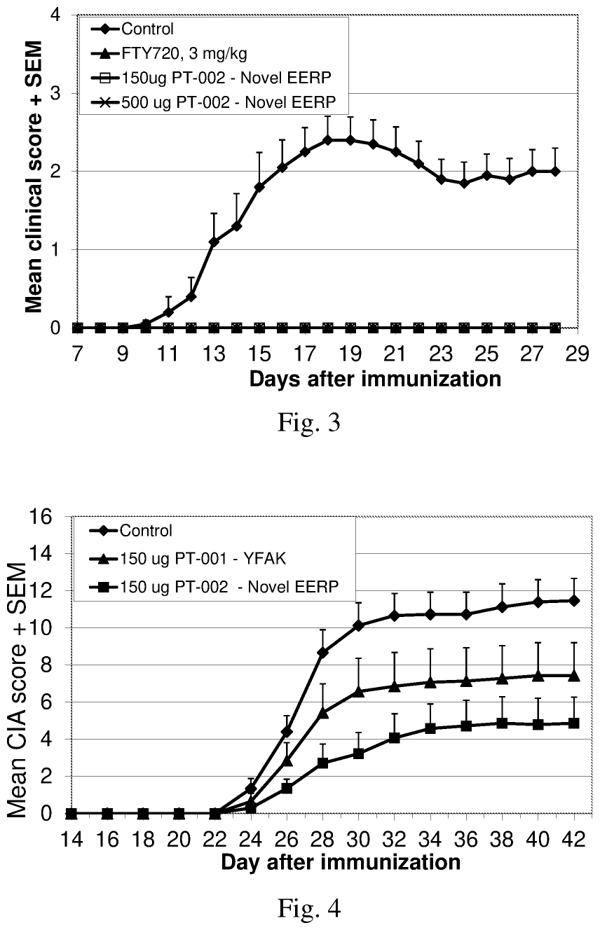

Prophylactic treatment of EAE, a mouse model of MS, by 2 different doses of EERP. EAE was induced in C57B 1/6 female mice (Taconic Biosciences) at 10 weeks of age, as described above, by MOG₃₅₋₅₅ (Control, depicted as diamonds) and treated prophylactically by co-administration of MOG₃₅₋₅₅ and FTY720 (fingolimod, Gilenya, depicted as triangles), MOG₃₅₋₅₅ and PT-002 (depicted as open squares), or MOG₃₅₋₅₅ and PT-002 (depicted as cross) at 150 μg/dose and 500 μg/dose, respectively (FIG. 3). Mice (10 per treatment group) were monitored daily in a blind fashion starting from Day 7 for signs of disease for 28 days post immunization. In contrast to the Control group, mice administered with FTY720 as well as the group treated with PT-002 did not exhibit any signs of disease. Study was performed by Hooke Labs (Lawrence, MA).

### Example 4

Prophylactic treatment of collagen-induced arthritis (CIA), a mouse model for rheumatoid arthritis (RA). CIA was induced in DBA/1 male mice at 8 weeks of age (Taconic Biosciences), acclimated for 3 weeks prior to initiation of the study, by immunization with bovine collagen in CFA (Control, depicted as diamonds) and treated prophylactically by co-administration of bovine collagen and PT-001 (depicted as triangles), or collagen and PT-002 (depicted as squares) at 150 μg/dose each (FIG. 4). On Day 18 after the first immunization, mice received a booster immunization with bovine collagen in CFA. All immunizations were subcutaneous into tails. Mice (15 per treatment group) were monitored in a blind fashion for signs of disease starting from Day 14 every other day for 42 days post immunization. PT-002 was the most effective at inhibiting clinical signs of disease as compared to PT-001 or Control. Study was performed by Hooke Labs (Lawrence, MA).

### PATENT LITERATURE

1. Fridkis-Hareli M, Strominger J L. Copolymers for suppression of autoimmune diseases, and methods of use. Norvegian Patent #337092. Issued Jan. 18, 2016.
2. Fridkis-Hareli M, Strominger J L. Copolymers for suppression of autoimmune diseases, and methods of use. Canadian Patent #CA 2768340. Issued Jun. 30, 2015.
3. Fridkis-Hareli M, Strominger J L. Synthetic peptides and methods of use for autoimmune disease therapies. U.S. Pat. No. 9,066,905. Issued Jun. 30, 2015.
4. Fridkis-Hareli M, Strominger J L. Copolymers for suppression of autoimmune diseases, and methods of use. U.S. Pat. No. 9,028,835 B2. Issued May 12, 2015. (another title from the Harvard letter: Novel synthetic copolymers that inhibit autoantigen-specific T cell responses and suppress EAE in SJL/J mice).
5. Fridkis-Hareli M, Strominger J L. Copolymers for suppression of autoimmune diseases, and methods of use. Japanese Patent #JP5525749. Issued Apr. 18, 2014.
6. Fridkis-Hareli M, Strominger J L. Copolymers for suppression of autoimmune diseases, and methods of use. Japanese Patent #JP2012162545. Issued Aug. 30, 2012.
7. Fridkis-Hareli M, Strominger J L. Copolymers for suppression of autoimmune diseases, and methods of use. Canadian Patent #CA 2614171. Issued Apr. 24, 2012.
8. Fridkis-Hareli M, Strominger J L. Copolymers for suppression of autoimmune diseases, and methods of use. U.S. Pat. No. 8,017,125. Issued Sep. 13, 2011.
9. Fridkis-Hareli M, Strominger J L. Linear random copolymers for suppression of autoimmune diseases and methods of use. Israeli Patent #161,121. Issued Mar. 1, 2011. (another title from the Harvard letter: Novel synthetic copolymers that inhibit autoantigen-specific T cell responses and suppress EAE in SIL/J mice).
10. Fridkis-Hareli M, Strominger J L. Synthetic peptides and methods of use for autoimmune disease therapies. U.S. Pat. No. 7,566,767. Issued Jul. 28, 2009.
11. Fridkis-Hareli M, Strominger J L. Therapeutic peptides for demyelinating conditions. Israeli Patent #157,073. Issued Mar. 30, 2009.
12. Fridkis-Hareli M, Strominger J L. Therapeutic peptides for demyelinating conditions. U.S. Pat. No. 7,456,252. Issued Nov. 25, 2008.
13. Sela M, Fridkis-Hareli M, Strominger J L, Aharoni R, Teitelbaum D, Arnon R. Treatment of autoimmune conditions with Copolymer 1 and related Copolymers. U.S. Pat. No. 7,425,332. Issued Sep. 16, 2008.
14. Fridkis-Hareli M, Strominger J L. Copolymers for suppression of autoimmune diseases, and methods of use. U.S. Pat. No. 7,381,790. Issued Jun. 3, 2008. (another title from the Harvard letter: Novel synthetic copolymers that inhibit autoantigen-specific T cell responses and suppress EAE in SIL/J mice).
15. Aharoni R, Teitelbaum D, Arnon R, Sela M, Fridkis-Hareli M, Strominger J L. Treatment of autoimmune conditions with Copolymer 1 and related Copolymers. U.S. Pat. No. 7,279,172. Issued Oct. 9, 2007.
16. Fridkis-Hareli M, Strominger J L. Copolymers for suppression of autoimmune diseases, and methods of use. Australian Patent #2002362445. Issued Jan. 11, 2007.
17. Fridkis-Hareli M, Strominger J L. Synthetic peptides for demyelinating conditions. U.S. Pat. No. 6,930,168. Issued Aug. 16, 2005.
18. Aharoni R, Teitelbaum D, Arnon R, Sela M, Fridkis-Hareli M, Strominger J L. Treatment of autoimmune conditions with Copolymer 1 and related Copolymers and Peptides. Hong Kong Patent 1038236. Issued Mar. 8, 2002.
19. Fridkis-Hareli M, Strominger J L. Novel synthetic copolymers that inhibit autoantigen-specific T cell responses and suppress EAE in SJL/J mice. U.S. Patent #60/326705. Issued Oct. 3, 2001.
20. Fridkis-Hareli M, Strominger J L. Novel synthetic copolymers that inhibit autoantigen-specific T cell responses and suppress EAE in SJL/J mice. PCT/US02/31399. Issued Oct. 3, 2002.
21. Strominger J L. Peptides for treatment of autoimmune diseases. PCT 2006/031727 A3. Published Mar. 23, 2006.
22. James Rasmussen, Jianxin Zhang, Sam Baldwan, Eric Zanelli, Bei Yu, Dustan Bonnin, Keith Johnson, Jeff Krieger. Methods of treating disease with random copolymers. US 20060194725 A1. U.S. patent Ser. No. 11/283,405.
23. James Rasmussen, Jianxin Zhang, Sam Baldwin, Eric Zanelli, Bei Yu, Dustan Bonnin, Keith Johnson. METHODS OF TREATING DISEASE WITH RANDOM COPOLYMERS. Pub. No.: US 2010/0316714 A1. Pub. Date: Dec. 16, 2010.
24. James Rasmussen, Jianxin Zhang, Sam Baldwin, Eric Zanelli, Bei Yu, Dustan Bonnin, Keith Johnson. METHODS OF TREATING DISEASE WITH RANDOM COPOLYMERS. CA02565819. Published Dec. 1, 2005.
25. Bonnin D, Zanelli E, Mathers T. Synthesis of directed sequence polymer compositions and antibodies thereof for the treatment of protein conformational disorders. Patent U.S. Pat. No. 8,546,532 B2. Date Oct. 1, 2013.
26. Bonnin D, Zanelli E, Mathers T. Directed sequence polymer compositions and antibodies thereof for the treatment of protein conformational disorders. US2014/0271614 A1. Published Sep. 18, 2014.
27. Bonnin D. Methods for designing and synthesizing directed sequence polymer compositions via the directed expansion of epitope permeability. U.S. Pat. No. 8,378,072, Issued Feb. 19, 2013.
28. Bonnin D. Methods for designing and preparing vaccines comprising directed sequence polymer compositions via the directed expansion of epitopes. Patent U.S. Pat. No. 9,533,037 Date Jan. 3, 2017.
29. Bonnin D, Zanelli E, Mathers T. Design and synthesis of directed sequence polymer compositions and antibodies thereof for the treatment of protein conformational disorders. PCT 2009/128948. Published Oct. 22, 2009.

### NON-PATENT LITERATURE

1. Braunwald E. et al., Principles of Internal Medicine, 15th edition, McGraw-Hill eds. (2001).
2. Rothbard, J. B., et al., 1991, Annu. Rev. Immunol. 9:527.
3. Gebe J. A. et al., Tissue Antigens 59:78 (2002).
4. Sakai K. et al., 1989. Proc. Natl. Acad. Sci. U.S.A. 86:9470
5. Hurtenbach U. et al., 1993. J. Exp. Med. 177:1499
6. Fairchild P. J. et al., 1994. Immunology 81:487
7. Brocke S. et al., 1996. Nature 379:343.
8. Sela, M., R. Arnon, et al., 1990. Bull. Inst. Pasteur (Paris) 88:303
9. Bornstein, M. B., et al., 1987, N. Engl. J. Med. 317:408
10. Johnson, K. P., et al., 1995, Neurology 45:1268
11. Fridkis-Hareli M, et al., 2002, Clin Invest. 109:1635
12. Fridkis-Hareli M, et al., 1998, Proc Natl Acad Sci U S A 95:12528
13. Fridkis-Hareli M, et al., 1999, Immunol. 162:4697
14. Fridkis-Hareli et al., 2001, Hum Immunol. 62:753
15. Fridkis-Hareli M, et al., 2000, Hum Immunol. 61:640
16. Erak M. et al., 2018, Bioorg. Med. Chem. 26:2759
17. Sherbet, G. British Journal of Medical Practitioners, 2009, 2:(1), 6
18. Getts, D R et al. Immunol Rev. 2013, 255(1):197
19. Rammensee H. G. et al., 1995. Immunogenetics 41:178
20. Stern L. J. et al., 1994. Nature 368:215
21. Ghosh P. et al., 1995. Nature 378:457
22. Dessen A. et al., 1997. Immunity 7:473
23. Lee K. H. et al., 2001. Nat Immunol. 2:501
24. http://hlaligand.ouhsc.edu and http://syfpeithi.bmi-heidelberg.com/scripts/MHCServer.dL1/home.htm
25. Sustained and Controlled Release Drug Delivery systems, J. R. Robinson, Ed., Marcel Dekker, Inc., NY, 1978

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. An amino acid copolymer having the sequence
(X)₀₋₁₉₅(Z)₅₋₂₀(X)₀₋₁₉₅
wherein X is one of tyrosine(Y), phenylalanine(F), alanine(A), and lysine(K); and Z consists of a peptide epitope that interacts with a major histocompatibility complex (MHC) class II protein and a T cell receptor.

2. The copolymer of claim 1, wherein the peptide epitope has the amino acid sequence AYKAA.

3. The copolymer of claim 1, wherein Y, F, A, and K are present in a molar ratio of about 0.01-3:0.01-5:2-30:1-10, respectively.

4. The copolymer of claim 1, wherein Y, F, A, and K are present in a molar ratio of about 0.1-2.5:0.1-5.0:2-25:2-8, respectively.

5. The copolymer of claim 1, wherein Y, F, A, and K are present in a molar ratio of about 0.8-2.0:0.1-4.0:2-20:2-6, respectively.

6. The copolymer of claim 1, wherein, Y, F, A, and K are present in a molar ratio of about 0.5-1.5:0.8-3.0:2-15:2-5, respectively.

7. The copolymer of claim 1, wherein Y, F, A, and K are present in a molar ratio of about 0.2-1.0:0.8-2.0:2-10:2-4, respectively.

8. The copolymer of claim 1, wherein Y, F, A, and K are present in a molar ratio of about 0.2-0.5:0.6-1.0:5-10:3-4, respectively.

9. The copolymer of claim 1, wherein Y, F A, and K are present in a molar ratio of about 0.2:0.5:5:3, respectively.

10. The copolymer of claim 1, comprising at least about 30 amino acid residues.

11. The copolymer of claim 1, comprising between about 30 and 200 and amino acid residues.

12. The copolymer of claim 1, wherein the amino acids are polymerized using a solid phase reaction.

13. The copolymer of claim 1, wherein the N-terminal amino acid is modified with an acetyl group.

14. The copolymer of claim 1, wherein the C-terminal amino acid is modified with an amide group.

15. The copolymer of claim 1, wherein one or more amino acids are modified to inhibit proteolytic degradation of the copolymer in a subject compared to a copolymer which is otherwise identical but lacking the amino acid modification.

16. A copolymer of claim 1, combined with at least one additional therapeutic agent.

17. The copolymer combined with an additional therapeutic agent according to claim 16 wherein the additional therapeutic agent is selected from the group consisting of an antibody, an enzyme inhibitor, an antibacterial, an antiviral, a steroid, a nonsteroidal anti-inflammatory, an antimetabolite, a cytokine, a cytokine blocking agent, an adhesion molecule blocking agent, and a soluble cytokine receptor.

18. The copolymer of claim 17, wherein the cytokine is selected from the group consisting of β-interferon, interleukin-4, and interleukin-10.

19. A composition comprising the copolymer of claim 1 and a pharmaceutically acceptable carrier.

20. A method of treating an immune-mediated disorder or an autoimmune disease in a subject in need thereof, the method comprising administering to the subject a therapeutically effective amount of the composition of claim 19.

21. The method of claim 20, wherein the immune-mediated disorder or an autoimmune disease is selected from the group consisting of ankylosing spondylitis and related spondyloarthropathies, autoimmune polyglandular syndrome, autoimmune Addison's disease, autoimmune alopecia, autoimmune hemolytic anemia, autoimmune thrombocytopenic purpura, acute rheumatic fever, age-related macular degeneration, celiac sprue, Crohn's disease, inflammatory bowel disease, dense deposit disease, dermatitis herpetiformis, Grave's disease, Goodpasture's syndrome, Guillain-Barre syndrome, Hashimoto's thyroiditis, immune-mediated infertility, insulin-dependent diabetes mellitus, insulin-resistant diabetes mellitus, multiple sclerosis, myasthenia gravis, neuromyelitis optica, pemphigus foliaceus, pemphigus vulgaris, rosacea, pernicious anemia, stiff-man syndrome, sympathetic ophthalmia, vitiligo, antiphospholipid syndrome, obstetrical antiphospholipid syndrome, rheumatoid arthritis, Sjogren's syndrome, systemic lupus erythematosus, systemic necrotizing vasculitis, autoimmune vasculitis, Wegener's granulomatosis, sepsis and septic shock, necrotizing enterocolitis, hemorrhagic fevers (e.g., Ebola, dengue and others), CFHR5 nephropathy, delayed graft-function, renal ischemia-reperfusion injury, IgA nephropathy (Berger's disease), antibody-mediated rejection, E-coli shiga toxin mediated disease (mostly kidney damage by toxin), atypical hemolytic uremic syndrome (aHUS, disseminated intravascular coagulation, deep vein thrombosis, warm autoimmune hemolytic anemia, Glanzmann's thrombasthenia, sickle cell anemia, preeclampsia, and asthma.

22. A kit comprising at least one unit dosage of the copolymer of claim 1.
